(19) United States
(12) Patent Application Publication
(10) Pub. No.: US 2008/0140028 A1
(43) Pub. Date: Jun. 12, 2008

(54) Mask for relief of dry eye syndrome

(76) Inventors: Mary Schwebel, Minneapolis, MN (US); Kays Chinai, Burlington, NJ (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/635,841

(22) Filed: Dec. 8, 2006

(51) Int. Cl. A61M 35/00; B65D 69/00; B29C 51/00; B29C 71/04

(52) U.S. Cl. 604/300; 206/570; 264/321; 264/232; 264/488; 264/294

(57) ABSTRACT

A mask to improve the comfort and eye health of individuals suffering from dry eye syndrome including a substantially vapor impermeable shell with a unitary structure having a substantially planar portion and two eye recesses. Each eye recess forms a generally concave dome shaped region on one side of the substantially planar portion. The shell is adapted to be worn on the face of the dry eye subject. An absorbent material can be positioned in at least one of the eye recesses; so that the absorbent material extends less than the maximum depth of the eye recesses to present the absorbent material to the face in the vicinity of the eyes of the dry eye subject without extending beyond the edge of the substantially planar portion. The invention may also include securing member to secure the shell to the subjects head.

### FIELD OF THE INVENTION

The invention relates to the treatment of dry eye syndrome. More particularly it relates to altering the local environment to reduce the discomfort of dry eye syndrome.

### BACKGROUND OF THE INVENTION

Masks for treatment of dry eye syndrome currently exist. Current masks for dry eye syndrome generally include a moisture impermeable and flexible enclosure and absorbent moisture reservoir, a seal, and a retaining strap or other retaining member. Currently available masks for the treatment of dry eye syndrome are designed to be used for a long period of time with the absorbent moisture reservoirs intended for repeated reuse.

Generally, currently available mask products are expensive. The expense of the currently available products encourages repeated reuse of the mask as well as the absorbent moisture reservoirs by users. Because the mask and moisture reservoirs are in close contact with the skin, the mask and/or moisture reservoirs may become contaminated with bacteria or other pathogens over time. The presence of bacteria or other pathogens creates a possibility of infection in or about the skin and the eyes. It is well known that a moist, dark environment provides a favorable environment for the growth of bacteria, fungi and other pathogens.

Currently available mask products may lack of sterility even at the initial use. The materials from which currently available products are made may tend to deteriorate under the conditions required for sterilization. Therefore, they are not provided in a sterile state. In addition, many of the materials are highly porous and may tend to absorb contaminants from the environment.

Thus, the treatment of dry eyes would benefit from a product that reduces the risks of infection and contamination.

### SUMMARY OF THE INVENTION

One embodiment of the present invention generally includes an eye mask, absorbent pads, and an eye care solution. The present invention solves many of the above discussed problems. The eye mask is soft, flexible, resilient and able to conform readily to the user's facial structure. The eye mask is non-permeable. It provides a vapor moisture barrier to contain moisture provided by the eye care solution. In addition, the eye mask of the invention is readily sterilizable by gamma radiation without deterioration. Further, the present invention lends itself to inexpensive production thus facilitating a disposable product which can be discarded after use to minimize the risk of contamination and infection.

In one embodiment, the eye mask of the present invention generally includes a shell, absorbent pads, and a retainer.

The shell generally includes a planar portion, domes, and walls.

In one aspect of the invention, the planar portion generally defines a brow arch, a nose relief, retainer ears, cheek arches, and dome perimeters. Brow arch is gently curved and extends generally between the retainer ears. The nose relief is centrally located on a lower edge of the shell and is formed by the juncture of the cheek arches.

In one aspect of the invention, the eye mask may be formed of closed cell flexible foam. Closed cell foam has excellent water vapor barrier properties. The product may be formed from polyolefin foams such as polyethylene foam. The eye mask is soft and flexible to degree that is not seen in prior masks, allowing the mask to conform to the shape of the face and ocular adnexa to sufficiently seal the mask to the face without the need for a separate sealing member.

For example, polyethylene foam sheets, such as those that can be obtained from Voltek of Lawrence, Mass. have been found to be suitable for use in the present invention. The following examples illustrate suitable qualities.

### EXAMPLE 1

foam sheet thickness 4.0 mm (Voltek of Lawrence, Mass., Code: LL1077);

density 0.035 grams per cc;

water vapor transmission rate 0.087 g/8 hours/100 sq inches-ASTM E96;

compression set 6% original thickness-ASTM D3575;

shore hardness 18 “A” scale; and

25% compression load 2.5 psi-ASTM D3575.

Such foam sheet material can be molded into a mask by heat and pressure. In this example the temperature of a mold was maintained at about 120° C. and the molding time was approximately 2.5 minutes.

### EXAMPLE 2

Polyethylene foam sheets (Voltek Code: LL1036):

Foam sheet thickness 3.4 mm;

Density 0.21 grams cm³;

Water vapor transition rate 0.087 g/8 hours/100 sq inches-ASTM E96;

Compression set3.8% original thickness;

Shore hardness 30 “A” scale; and

25% compression load 8.5 psi.

A mask produced from this material is less flexible than that described above in Example 1. These examples should not be considered limiting.

An absorbent pad for use with the eye mask generally should demonstrate a fast rate of absorption of liquids.

In one aspect of the invention, each pad may hold at least 15 grams of eye care solution under a 0.1 pound per square inch load. The absorbent pad may be lint free as much as possible. The absorbent pad, in one aspect of the invention, may be sterilizable by gamma radiation without observable damage. Finally the absorbent pad should have a low level of extractables. An example absorbent pad can be formed from pulp and super absorbent material combined with bonded non-woven materials, such as those obtained from Buckeye Technologies of Memphis, Tenn. Example material specifications are as follows:

| | | Bonded |
|---|---|---|
| | | Nonwoven |
| | Pulp/SAP Blend | Material |
| Product Code | V3921 | V3027 |
| Basis Weight (g/m²) | 165.00 | 100.00 |
| Density (g/cc) | 0.09 | 0.05 |
| SAP Cont. (%) | 26.00 | 0.00 |
| Pulp (%) | 68.00 | 89.5 |
| Binder (%) | 6.00 | 10.5 |
| Type of SAP | Crosslinked | — |
| | poly Acrylate | |
| Residual Monomer (ppm) | <75 | — |

The absorbent pad also may include hydrotangled polyester-rayon blend fiber fabric such as that which can be obtained from PGI of Mooresville, N.C. This fabric may be used as a cover material.

In addition, the absorbent pad can include a 1 mil polyethylene film such as that obtained from Tedegar Film Products of Richmond, Va. In one embodiment of the invention, the absorbent pad bonded together by a hot melt adhesive such as that which can be obtained from HB Fuller of St. Paul, Minn.

Absorbent pads constructed from the above discussed materials displayed the following absorption properties:

absorption capacity at 0.1 psi load 16.8 grams;

rate of absorption 3.4 ml per second; and

### These values should not be considered limiting.

An exemplary eye care solution can include water, sterile water, saline solution and an aqueous solution containing electrolytes such as sodium chloride or potassium chloride. For example, a typical formulation would be:

sterile water 98.9%;

sodium chloride 0.5%;

potassium chloride 0.2%; and

glycerin 0.4%.

In this example, glycerin acts as a lubricant and controls the rate of moisture evaporation from the pad. The solution may be packaged, for example, in 20 ml plastic vials.

For distribution purposes, the mask, a quantity of absorbent pads, and an eye care solution vial can be packaged in a conventional plastic material such as polyester or polyethylene. The packaging may be sealed to be air tight.

The eye mask can be sterilized in a cardboard case by application of gamma radiation. Approximately a 20-30 kilogray radiation dosage is expected to be required to sterilize the product.

### BRIEF DESCRIPTION OF THE DRAWINGS

FIG. 1 is a partially exploded perspective view of a mask in accordance with the present invention.

FIG. 2 is rear plan view of the mask in accordance with the present invention.

FIG. 3 is a cross sectional view of an absorbent pad in accordance with the present invention taken along section line 3-3 of FIG. 2.

FIG. 4 is a perspective view of another embodiment of a mask in accordance with the present invention.

FIG. 5 is a rear plan view of the embodiment of the mask depicted in FIG. 4.

### DETAILED DESCRIPTION OF THE INVENTION

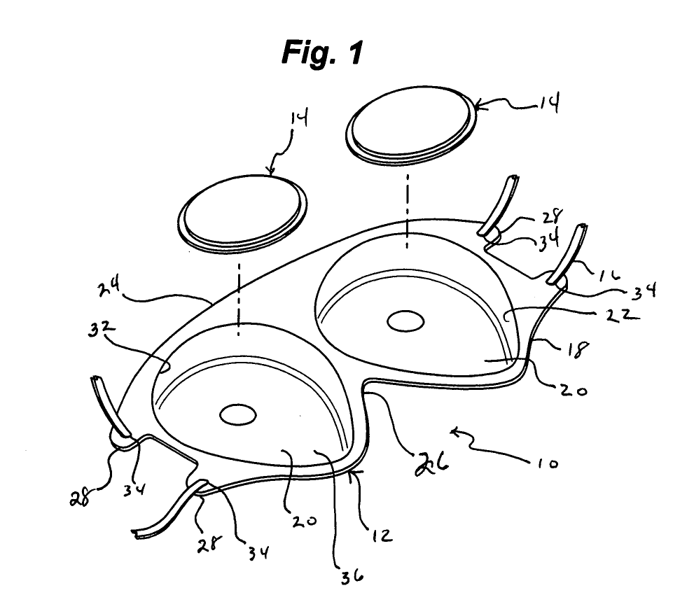

Referring to FIG. 1, in one embodiment, eye mask 10 of the present invention generally includes shell 12, absorbent pads 14, and retainer 16.

Shell 12 generally defines planar portion 18, domes 20, and walls 22.

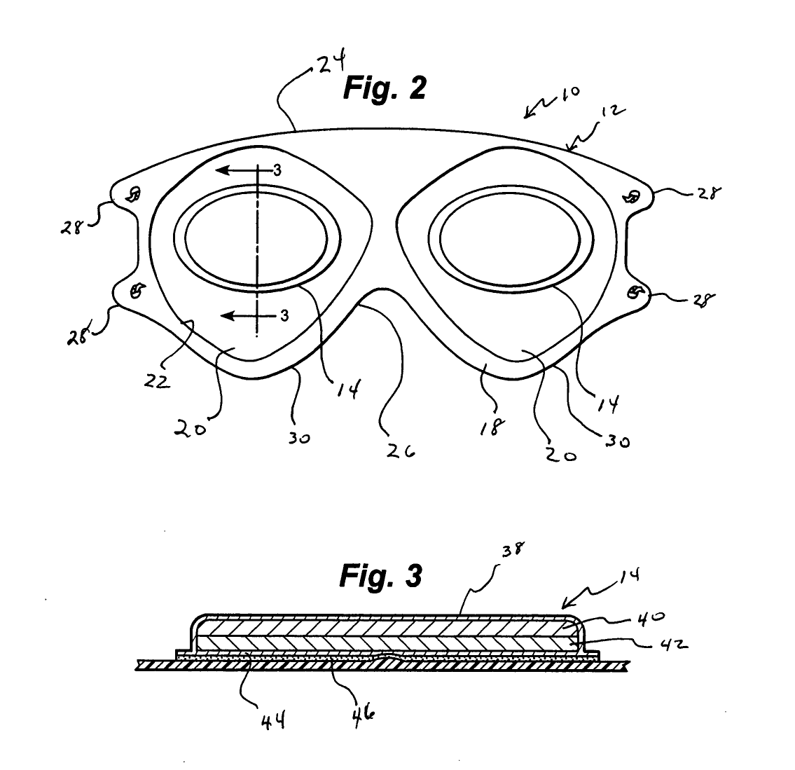

Referring to FIGS. 1 and 2, in one aspect of the invention, planar portion 18 generally defines brow arch 24, nose relief 26, retainer ears 28, cheek arches 30, and dome perimeters 32. Brow arch 24 is gently curved and stretches generally between retainer ears 28. Nose relief 26 is centrally located on a lower edge of shell 12 and is formed by the juncture of cheek arches 30. Retainer ears 28 may define apertures 34 therein for receiving retainers 16.

Referring again to FIGS. 1 and 2, domes 20 together with walls 22 define eye recesses 36. Eye recesses 36 may take the shape of roughly an irregular pentagon. Eye recesses 36 have a central depth sufficient to allow absorbent pads 14 to be inserted therein while still leaving sufficient space in front of the user's eyes for an air gap between absorbent pads 14 and user's eyelids. For example, eye recesses may have a depth of approximately 1-2 cm.

Referring particularly to FIG. 3, absorbent pads 14 generally include vapor permeable cover 38, bonded non-woven layer 40, pulp super absorbent polymer (SAP) blend 42, vapor barrier 44, and securing member 46. Vapor permeable cover 38 may be formed of a fabric or non-fabric material which allows the passage of water vapor and related vapors. Vapor permeable cover 38 may be formed, for example, from hydrotangled polyester rayon blend fiber fabric of the type that can be obtained from PGI, Mooresville, N.C.

Bonded non-woven layer 40 may be formed from an absorbent non-woven material such as that available under product code V3027 from Buckeye Technologies, Memphis, Tenn. The bonded non-woven material may have, for example, a basis weight of approximately 100 grams per square meter, a density of approximately 0.05 grams per cubic centimeter and be formed of approximately 89.5% pulp and 10.5% binder.

Pulp super absorbent polymer (SAP) blend 42 may be, for example, that available under product code V3921 also from Buckeye Technologies. This material may, for example, have a basis weight of approximately 165 grams per square meter, a density of about 0.09 grams per cc, a super absorbent polymer content of approximately 26%, a pulp content of approximately 68%, and a binder content of approximately 6%. Pulp SAP blend 42 may include a super absorbent polymer of, for example, cross linked polyacrlylate having a residual monomer of less than 75 parts per million.

Vapor barrier 44 may be formed from, for example, 1 mil polyethylene film such as that which can be obtained from Tredegar Film Products of Richmond, Va. Vapor permeable cover 38 may be secured to vapor barrier 44 around a common perimeter thereof by use of, for example, hot melt adhesive or another adhesive product. Vapor permeable cover 38 may also be connected to vapor barrier 44 by mechanical or other fastening approaches.

Securing member 46 serves to secure vapor barrier 44 to shell 12. Desireably, securing member 46 can be secured and removed at least several times. For example, securing member 46 may include a hook and loop system or an adhesive which can be repeatedly placed and removed. Securing member 46 may be placed into permanent contact with vapor barrier 44 or the interior of domes 20, for example.

Absorbent pads 14 may be moistened with an eye care solution. The eye care solution may be an aqueous solution containing electrolytes. Eye care solutions can include water, sterile water, saline solution and an aqueous solution containing electrolytes such as sodium chloride or potassium chloride. For example, a formulation may include sterile water 98.9%, sodium chloride 0.5%, potassium chloride 0.2%, and glycerin 0.4%. The eye care solution may also be any other aqueous solution amenable to maintaining a healthy humidity environment for the eyes. In addition, plain distilled water may be used to moisturize the absorbent pads 14.

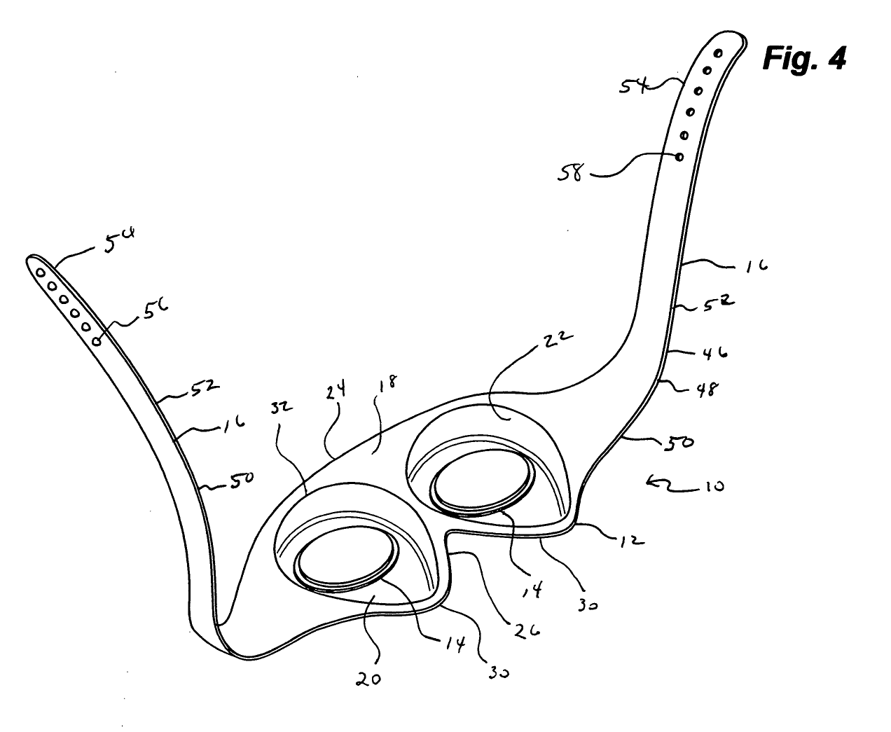

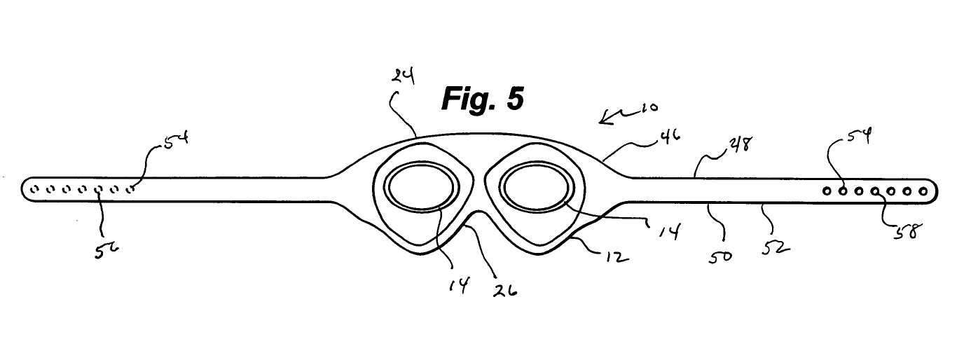

Referring to FIGS. 4 and 5 in another embodiment, eye mask 10 generally includes planar portion 18 and domes 20 similar to those described above.

In this embodiment, eye mask 10 includes integral retainers 48. Integral retainers 48 may form an extension 50 of planar portion 18.

Integral retainers 48 may include straps 52 and coupling members 54. Coupling members 54 may include any sort of coupling known in the art. In this example, coupling members 54 include buttons 56 and apertures 58. Buttons 56 may be integrally molded to straps 52 and protrude therefrom. Apertures 58 to receive buttons 56 are formed in an opposing strap 52. Buttons 56 and apertures 58 are desirably sized so that buttons 56 may be forced through apertures 58 and retained therein, thus coupling straps 52 to each other to secure eye mask 10 about the head of the user. Other coupling members 54 may of course be used. For example, coupling members 54 may include tabs and slots or other means known to the art to couple two straps 52 together.

In operation, eye mask 10 is supplied in a kit including shell 12, absorbent pads 14, retainer 16, and eye care solution (not shown.) Prior to distribution, eye mask 10 is sterilized with gamma radiation or by other sterilization techniques. About a 20-30 kilogray dosage is expected to be required to sterilize the product.

When a user is ready to use an eye mask 10. The user removes shell 12 and absorbent pads 14 from packaging. The user secures absorbent pads 14 to the interior of domes 20 then applies the eye care solution to absorbent pads 14. The user then places eye mask 10 over the eyes and face and secures it in place with retainer 16 or integral retainers 48.

Eye mask 10 with absorbent pads 14 in place, may be used during sleep or during the day for any period of time desired.

Eye care solution evaporates from absorbent pads 14 to provide a humidity rich environment within eye recesses 36, thus, reducing evaporation of tears and providing additional humidity to provide comfort and therapeutic benefits to the dry eye patient.

For example, polyethylene foam sheets, such as those that can be obtained from Voltek of Lawrence, Mass. have been found to be suitable for use in the present invention. The following examples illustrate suitable qualities.

### EXAMPLE 1

foam sheet thickness 4.0 mm (Voltek of Lawrence, Mass., Code: LL1077);

density 0.035 grams per cc;

water vapor transmission rate 0.087 g/8 hours/100 sq inches-ASTM E96;

compression set 6% original thickness-ASTM D3575;

shore hardness 18 “A” scale; and

25% compression load 2.5 psi-ASTM D3575.

Such foam sheet material can be molded into a mask by heat and pressure. In this example the temperature of a mold was maintained at about 120° C. and the molding time was approximately 2.5 minutes.

### EXAMPLE 2

Polyethylene foam sheets (Voltek Code: LL1036):

Foam sheet thickness 3.4 mm;

Density 0.21 grams cm³;

Water vapor transition rate 0.087 g/8 hours/100 sq inches-ASTM E96;

Compression set 3.8% original thickness;

Shore hardness 30 “A” scale; and

25% compression load 8.5 psi.

A mask produced from this material is less flexible than that described above in Example 1.

An absorbent pad for use with the eye mask generally should demonstrate a fast rate of absorption of liquids.

In one aspect of the invention, the pad may hold at least 15 grams of eye care solution under a 0.1 pound per square inch load. The absorbent pad may be lint free as much as possible. The absorbent pad, in one aspect of the invention, may be sterilizable by gamma radiation without observable damage. Finally, the absorbent pad should have a low level of extractables. An example absorbent pad can be formed from pulp and super absorbent material combined with bonded non-woven materials, such as those obtained from Buckeye Technologies of Memphis, Tenn. Example material specifications are as follows:

| | | Bonded |
|---|---|---|
| | | Nonwoven |
| | Pulp/SAP Blend | Material |
| Product Code | V3921 | V3027 |
| Basis Weight (g/m²) | 165.00 | 100.00 |
| Density (g/cc) | 0.09 | 0.05 |
| SAP Cont. (%) | 26.00 | 0.00 |
| Pulp (%) | 68.00 | 89.5 |
| Binder (%) | 6.00 | 10.5 |
| Type of SAP | Crosslinked | — |
| | poly Acrylate | |
| Residual Monomer (ppm) | <75 | — |

The absorbent pad also may include hydrotangled polyester-rayon blend fiber fabric such as that which can be obtained from PGI of Mooresville, N.C. This fabric may be used as a cover material.

In addition, the absorbent pad can include a 1 mil polyethylene film such as that obtained from Tedegar Film Products of Richmond, Va. In one embodiment of the invention, the absorbent pad bonded together by a hot melt adhesive such as that which can be obtained from HB Fuller of St. Paul, Minn.

Absorbent pads constructed from the above discussed materials displayed the following absorption properties:

absorption capacity at 0.1 psi load 16.8 grams;

rate of absorption 3.4 ml per second; and

### These values should not be considered limiting.

An exemplary eye care solution can be an aqueous solution containing electrolytes. For example, a typical formulation would be:

sterile water 98.9%;

sodium chloride 0.5%;

potassium chloride 0.2%; and

glycerin 0.4%.

In this example, glycerin acts as a lubricant and controls the rate of moisture evaporation from the pad. The solution may be packaged in 20 ml plastic vials.

For distribution purposes, the mask, a quantity of absorbent pads, and an eye care solution vial can be packaged in a conventional plastic material such as polyester or polyethylene. The packaging may be sealed to be air tight.

The eye mask can be sterilized in a cardboard case by application of gamma radiation. Approximately a 20-30 kilogray radiation dosage is expected to be required to sterilize the product.

### EXAMPLE

A total of 22 subjects reporting a mild to moderate dry eye condition were impaneled to evaluate an embodiment of the invention over 4 weeks of nightly use. A total of 21 subjects completed the study. One subject discontinued study participation for reasons unrelated to use of the invention.

Following the 4-week use period, subjects completed a computerized questionnaire regarding the invention's performance and attributes. Subjects choosing a neutral response (“neither agree nor disagree” or “not sure”) were removed from the questionnaire summary. The majority of the test population reporting an opinion indicated the following: the embodiment was easy to use, fit on the face, was comfortable on the face, improved the dry eye problem and had a favorable appearance; the eye pads provided enough humidification during use; they liked the quality and were satisfied with the embodiment. The majority of subjects reporting a preference indicated they would purchase the embodiment if it was available.

### Panel Selection

A total of 22 healthy female subjects, ranging in age from 18 to 61 years, were selected for the study. Seven subjects (approximately 32%) wore contact lenses and 15 subjects (approximately 68%) were non-contact lens wearers.

### Test Method

On the first day of the study, each subject completed a baseline questionnaire evaluating dry eye symptoms. Qualified subjects were assigned sequential subject numbers in the order of their qualification and were issued an identically numbered test kit. Subjects were given a copy of the sponsor's use directions and panelist instructions and were directed to use the test material every night at home for 4 weeks. Other than the assigned test material, the panelists were to introduce no new cosmetics during the study. Subjects were permitted to use their usual over-the-counter moisturizing eye drops as needed during the day.

Subjects used the test materials as instructed and completed a weekly questionnaire after each week of use.

After the 4-week use period, subjects returned the test materials and weekly use evaluations and responded to a computerized questionnaire.

### Questionnaires

Final questionnaire responses

The majority of the test population reporting an opinion indicated the following qualities or test material effects:

The eye care device was easy to use.

The eye care device fit on the face.

The eye care device was comfortable on the face.

The eye pads provided enough humidification during use. The eye care device improved the dry eye problem.

The eye care device had a favorable appearance.

Subjects liked the quality of the eye care device.

Subjects were satisfied with the eye care device.

Given the opportunity, the subjects would purchase the eye care device.

The present invention may be embodied in other specific forms without departing from the spirit of the essential attributes thereof; therefore, the illustrated embodiments should be considered in all respects as illustrative and not restrictive, reference being made to the appended claims rather than to the foregoing description to indicate the scope of the invention.

1. A mask to improve the comfort and eye health of dry eye subjects, comprising:
a substantially vapor impermeable shell comprising a unitary structure having a substantially planar portion and two eye recesses each forming a generally concave dome shaped region on one side of the substantially planar portion, the shell being adapted to be worn on the face of the dry eye subject, each eye recess having a concave side;
an absorbent material operably positionable in at least one of the eye recesses; such that the absorbent material extends less than a maximum depth of the eye recess so as to present the absorbent material to the face of the dry eye subject without extending beyond an edge of the substantially planar portion; and
a securing member to secure the shell to the subjects head.

2. The mask as claimed in claim 1, wherein the shell is adapted to be sterilized with gamma radiation substantially without deterioration.

3. The mask as claimed in claim 1, wherein the securing member comprises integral retainers that are part of the unitary structure and which extend outwardly from the shell whereby the shell can be secured to a head of the subject.

4. The mask as claimed in claim 3, wherein the integral retainers include a first integral retainer, a second integral retainer and a coupling member whereby the first integral retained and the second integral retainer may be coupled together to secure the shell to the head of the subject.

5. The mask as claimed in claim 1, wherein the absorbent pad comprises a super absorbent polymer.

6. The mask as claimed in claim 1, wherein the absorbent pad comprises a vapor permeable cover portion and a vapor barrier cover portion.

7. The mask as claimed in claim 1, wherein the absorbent pad comprises a securing member by which it is securable to the shell.

8. The mask as claimed in claim 1, wherein the shell is formed from a sheet of molded foam material.

9. A kit to improve the comfort and eye health of dry eye subjects, comprising:
a mask comprising a substantially vapor impermeable shell comprising a unitary structure having a substantially planar portion and two eye recesses each forming a generally concave dome shaped region on one side of the substantially planar portion, the shell being adapted to be worn on the face of the dry eye subject, each eye recess having a concave side;
an absorbent material operably positionable in at least one of the eye recesses; such that the absorbent material extends less than a maximum depth of the eye recess so as to present the absorbent material to the face of the dry eye subject without extending beyond an edge of the substantially planar portion;
a securing member to secure the shell to the subjects head
a supply of an eye care liquid; and
wherein at least the mask and the absorbent material are supplied in a sterile state in a substantially sealed container.

10. The kit as claimed in claim 9, wherein the shell is adapted to be sterilized with gamma radiation substantially without deterioration.

11. The kit as claimed in claim 9, wherein the securing member comprises integral retainers that are part of the unitary structure and which extend outwardly from the shell whereby the shell can be secured to a head of the subject.

12. The kit as claimed in claim 9, wherein the integral retainers include a first integral retainer, a second integral retainer and a coupling member whereby the first integral retained and the second integral retainer may be coupled together to secure the shell to the head of the subject.

13. The kit as claimed in claim 9, wherein the absorbent pad comprises a super absorbent polymer.

14. The kit as claimed in claim 9, wherein the absorbent pad comprises a vapor permeable cover portion and a vapor barrier cover portion.

15. The kit as claimed in claim 9, wherein the absorbent pad comprises a securing member by which it is securable to the shell.

16. A method of manufacturing a mask to improve the comfort and eye health of dry eye subjects, comprising:
cutting out a unitary portion of a foam sheet material;
molding the unitary portion of a foam sheet material under heat and pressure to form a mask having a substantially planar portion and two eye recesses each eye recess having a concave side.

17. The method claimed in claim 16, further comprising sterilizing the shell.

18. The method claimed in claim 17, further comprising utilizing gamma radiation to sterilize the shell.

19. The method claimed in claim 16, further comprising forming integral retainers that are part of the unitary structure and which extend outwardly from the shell.

20. The method claimed in claim 16, further comprising forming a first integral retainer, a second integral retainer and a coupling member.

21. An eye mask for the relief of symptoms of dry eye syndrome, comprising:
a front portion formed of flexible closed cell foam having a density ranging from about 0.025 grams per cubic centimeter to about 0.25 grams per cubic centimeter and a Shore hardness of about 15 to about 35 on the A scale.
an absorbent pad capable of holding at least about 10 grams of eye care solution under a 0.1 pound per square inch load; and
a securing member to secure the front portion to the subjects head.

22. The mask as claimed in claim 21, wherein the absorbent pad comprises a super absorbent polymer.

23. The mask as claimed in claim 21, wherein the absorbent pad comprises a vapor permeable cover portion and a vapor barrier cover portion.
